(19) United States
(12) Patent Application Publication
(10) Pub. No.: US 2006/0150977 A1
(43) Pub. Date: Jul. 13, 2006

(54) Respiratory assist device and method of providing respiratory assistance

(76) Inventor: Arthas Flabouris, South Australia (AU)

Correspondence Address: MEDLEN & CARROLL, LLP, 101 HOWARD STREET, SUITE 350, SAN FRANCISCO, CA, 94105, US

(21) Appl. No.: 10/542,525

(22) PCT Filed: Jan. 16, 2004
(86) PCT No.: PCT/AU04/00050

§ 371 Date: Jan. 17, 2006

(30) Foreign Application Priority Data
Jan. 17, 2003 (AU) 2003900203

(51) Int. Cl. A61M 16/00; A62B 7/10

(52) U.S. Cl. 128/205.28; 128/205.25; 128/910; 128/205.12; 128/204.25

(57) ABSTRACT

A respiratory assist device (10) adapted for transport use. The device (10) including a venturi means (16), a means (36) to deliver gas to a patient's airways, and a Carbon Dioxide absorber (44) in a gas circuit (12) having at least a breathable gas mixture of fresh gas and Carbon Dioxide depleted gas travelling therethrough. The venturi means (16) is adapted to receive fresh gas from a fresh gas source (18) and entrain the fresh gas into the gas mixture in the circuit (12), thereby increasing the velocity of the gas mixture in the circuit (12). The delivery means (36) is downstream of the venturi means (16) and is also adapted to deliver the gas mixture from the circuit (12) to a patient's airways and return the patient's exhaled gas to the circuit (12). The Carbon Dioxide absorber (44) is downstream of the delivery means (36) and upstream of the venturi means (16). The absorber (44′) is adapted to remove Carbon Dioxide from the gas mixture in the circuit (12).

### FIELD OF THE INVENTION

The present invention relates to a respiratory assist device and a method of providing respiratory assistance, the device/method being adapted for transport use.

The invention has been primarily developed for transport use, particularly transport of patients to hospital (ie. pre hospital use) and transport of patients between and within hospitals, including emergency transportation such as land and air ambulances, and will be described hereinafter with reference to this application. However, it will be appreciated that the invention is not limited in this particular field of use and is also suitable for use in other out of hospital environments such as remote locations (eg. mines, altitude, submariner) in which decompression illness may arise and environments that may generate acute inhalational lung injury through heat, smoke, and (in the presence of fires) inhalational toxins.

The primary use of the invention is in providing non-invasive respiratory assistance via a face mask. However, the invention can also be adapted for use with intubated patients receiving either mandatory or assisted respiratory support. As used herein, ‘mandatory’ refer to patients in whom the device is delivering all the respiratory effort and ‘assisted’ refers to patients who are making some respiratory effort but that effort is being assisted by the device.

### BACKGROUND OF THE INVENTION

There are presently two main types of respiratory assist devices. The first type are devices that generate/deliver continuous positive airway pressure (CPAP), which are generally used in the home treatment of sufferers of sleep apnea and other sleep disordered breathing conditions. The main disadvantage of such machines that are used to deliver CPAP in a home, pre-hospital or emergency environment is they are not suitable for delivering breathable gas with a high concentration of oxygen, as is often required for patients with compromised breathing due to illness or trauma. Machines that deliver CPAP also generally require a 240 volt power supply, can be noisy and can be cumbersome in transport environments. Machines that are used to deliver CPAP in a hospital environment can deliver higher levels of oxygen but have high gas consumption. This means they typically require attachment to a fixed wall outlet and/or to large gas cylinders that cannot easily be used during transportation of patients, other than distances that are far less than the usual practice.

The other main type of respiratory assist devices are mechanical ventilators, as often used in intensive care units of hospitals. The main disadvantage of such ventilators is that they require a tube to be placed into the patient's trachea to deliver gas directly to the patient's lungs. This process is known as intubation and can itself place the patient at risk due to airway trauma during intubation, acute cardio-respiratory effects of mandatory positive pressure ventilation, the requirement for an anaesthetic and continual sedation, and greater complexity of ventilatory support. In cases of extended use (for example more than 48 hours), ventilators can lead to the patient developing pneumonia. Another disadvantage of ventilators is that their gas consumption is too high for use with portable gas supplies (eg. gas cylinders) for treatment times in excess of about 20 minutes. They are also very complicated to operate, are expensive and are not suited to transport environments. Some such ventilators may also deliver CPAP with higher levels of oxygen, but have the same limitations as those devices outlined above.

### OBJECT OF THE INVENTION

It is the object of the present invention to substantially overcome or at least ameliorate one or more of the above noted prior art disadvantages.

### SUMMARY OF THE INVENTION

Accordingly, in a first aspect, the present invention provides a respiratory assist device adapted for transport use, the device including the following components in a gas circuit having at least a breathable gas mixture of fresh gas and Carbon Dioxide depleted gas travelling therethrough:

a venturi means adapted to receive fresh gas from a fresh gas source and entrain the fresh gas into the gas mixture in the circuit, thereby increasing the velocity of the gas mixture in the circuit;

means to deliver the gas mixture to the patient's airways, the delivery means being downstream of the venturi means and adapted to deliver the gas mixture from the circuit to a patient's airways and return the patient's exhaled gas to the circuit; and

a Carbon Dioxide absorber downstream of the delivery means, and upstream of the venturi means, and adapted to remove Carbon Dioxide from the gas mixture in the circuit.

In a non-invasive form, the delivery means is preferably in the form of a face mask. In an invasive form, the delivery means is preferably in the form of a tracheal tube.

The venturi means is preferably adapted to receive the fresh gas, which is most preferably Oxygen or Oxygen enriched, from a bottled or piped supply, desirably via a blender and an adjustable gas flow meter. In a preferred form, the venturi means is a jet delivery nozzle.

The Carbon Dioxide absorber desirably also includes a water trap.

The device preferably also includes a first unidirectional flow device in gas communication with the circuit between the venturi means and the delivery means, the first unidirectional flow device adapted to only permit gas flow from the venturi means towards the delivery means. The first unidirectional flow device desirably includes a fan or other device adapted to increase the velocity of the breathable gas mixture in the circuit.

The device preferably also includes a second unidirectional flow device in gas communication with the circuit between the delivery means and the Carbon Dioxide absorber, the second unidirectional flow device adapted to only permit gas flow from the delivery means towards the Carbon Dioxide absorber.

The device preferably also includes a gas reservoir in gas communication with the circuit between the Carbon Dioxide absorber and the venturi means. The gas reservoir is desirably in the form of a compliant bag, most desirably with a capacity of 500-2000 mls. The interior of the bag is preferably in gas communication with the circuit.

In the non-invasive form, the exterior of the bag is preferably adapted, upon sufficient inflation, to block a supplemental gas flow chamber from communicating with the circuit and, in the absence of sufficient inflation, to allow the supplemental gas flow chamber to communicate with the circuit. The supplemental gas flow chamber is preferably connected to the circuit via at least one third unidirectional flow device adapted to only permit gas flow from the supplemental gas flow chamber towards the circuit. The third uni-directional flow device is most preferably a pair of flap valves. The supplemental gas flow chamber is preferably supplied with gas from the blender, most preferably via a pressure reducing valve, an adjustable pressure regulating valve and an over pressure relief valve.

In the invasive form, the exterior of the bag is located in a sealed chamber adapted for pressurisation by a ventilator. In one arrangement, the ventilator is gas powered. In another arrangement, the ventilator is electrically powered. The ventilator is actuated to pressurise the chamber and thereby cause the volume of the gas in the bag to be added to the circuit.

The device preferably also includes one or more of: a circuit pressure monitor and associated alarm; an Oxygen analyser; a Carbon Dioxide analyser; a safety valve adapted to open the circuit to atmosphere upon sensing that the fresh gas supply is exhausted; and a device adapted to servo control the fresh gas flow delivery.

In a second aspect, the present invention provides a method of providing respiratory assistance adapted for a transport environment, the method including performing the following steps in a gas circuit having at least a breathable gas mixture of fresh gas and Carbon Dioxide depleted gas travelling therethrough:

entraining fresh gas into the circuit, thereby increasing the velocity of the gas mixture in the circuit;

delivering the gas mixture, downstream of the fresh gas entrainment, from the circuit to a patient's airways and returning the patient's exhaled gas to the circuit; and

absorbing Carbon Dioxide from the circuit downstream of the patient's airways and upstream of the entrainment.

The method preferably also includes the step of receiving the fresh gas for entrainment, which is most preferably Oxygen or Oxygen enriched, from a bottled or piped supply, desirably after passing it through a gas blender that incorporates air and an adjustable gas flow meter. The entrainment is preferably performed with a venturi means, most preferably a jet delivery nozzle.

The method preferably also includes the step of absorbing water from the circuit downstream of the patient's airways and upstream of the entrainment, most preferably during the Carbon Dioxide absorption.

The method preferably also includes the step of allowing only unidirectional gas flow in the circuit from the venturi means towards the patient's airways. The method preferably also includes the step of increasing the velocity of the breathable gas mixture in the circuit, most preferably with a fan or the like.

The method preferably also includes the step of allowing only unidirectional gas flow in the circuit from the patient's airways towards the Carbon Dioxide absorber.

The method preferably also includes the step of providing supplemental gas flow to the circuit upon sensing that the gas flow in the circuit is not sufficient to meet the patient's respiratory needs.

In a third aspect, the present invention provides a respiratory assist device, the device comprising the following components in a gas circuit having at least a breathable gas mixture of fresh gas and Carbon Dioxide depleted gas travelling therethrough:

a venturi for receiving fresh gas from a fresh gas source and entraining the fresh gas into the gas mixture in the circuit, thereby increasing the velocity of the gas mixture in the circuit;

an airway gas delivery assembly downstream of the venturi for delivering the gas mixture from the circuit to a patient's airways and returning the patient's exhaled gas to the circuit; and

a Carbon Dioxide absorber downstream of the mask, and upstream of the venturi, for removing Carbon Dioxide from the breathable gas mixture in the circuit.

In a fourth aspect, the present invention provides a method of providing respiratory assistance in a gas circuit having at least a breathable gas mixture of fresh gas and Carbon Dioxide depleted gas travelling therethrough, the method comprising the following steps:

entraining fresh gas into the circuit to increase the velocity of the gas mixture in the circuit;

delivering the gas mixture, downstream of the fresh gas entrainment, from the circuit to a patient's airways and returning the patient's exhaled gas to the circuit; and

absorbing Carbon Dioxide from the circuit downstream of the patient's airways and upstream of the entrainment.

In a fifth aspect, the present invention provides a respiratory assist device comprising:

a gas circuit having at least a breathable gas mixture of fresh gas and Carbon Dioxide depleted gas travelling therethrough;

a venturi for receiving fresh gas from a fresh gas source and entraining the fresh gas into a gas mixture in the circuit, thereby increasing the velocity of the gas mixture in the circuit;

an airway gas delivery assembly downstream of the venturi for delivering the gas mixture from the circuit to a patient's airways and returning the patient's exhaled gas to the circuit; and

a Carbon Dioxide absorber downstream of the mask, and upstream of the venturi, for removing Carbon Dioxide from the breathable gas mixture in the circuit.

In a sixth aspect, the present invention provides a method of providing respiratory assistance in a gas circuit, the method comprising the following steps:

entraining fresh gas into the circuit to increase the velocity of a breathable gas mixture in the circuit, the gas mixture having the fresh gas and also Carbon Dioxide depleted gas travelling therein;

delivering the gas mixture, downstream of the fresh gas entrainment, from the circuit to a patient's airways and returning the patient's exhaled gas to the circuit; and

absorbing Carbon Dioxide from the circuit downstream of the patient's airways and upstream of the entrainment.

### BRIEF DESCRIPTION OF THE DRAWINGS

Preferred embodiments of the invention will now be described, by way of examples only, with reference to the accompanying drawings in which:

FIG. 1 is a schematic diagram of a first embodiment of a non-invasive respiratory assist device according to the invention;

FIG. 2 is a schematic view of a jet delivery nozzle used in the device shown in FIG. 1;

FIG. 3a is a schematic side view of a face mask used in the device shown in FIG. 1;

FIG. 3b is a partial schematic underside view of the face mask shown in FIG. 3a;

FIG. 3c is a partial schematic cross sectional side view of the face mask shown in FIG. 3a; and

FIG. 4 is a schematic diagram of a second embodiment of an invasive respiratory assist device according to the invention.

### DETAILED DESCRIPTION OF THE PREFERRED EMBODIMENTS

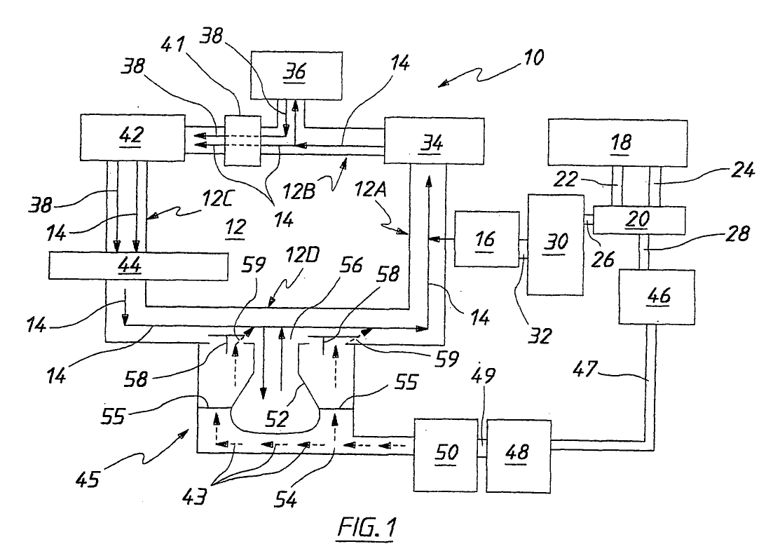

Referring firstly to FIG. 1, there is shown a schematic diagram of a first embodiment of a non-invasive respiratory assist device 10 adapted for transport use, particularly emergency patient transportation. The device 10 includes a number of components, as will be described in detail below, connected in a (substantially closed) gas circuit 12. The circuit 12 is divided into four main circuit sections (12a,b,c and d), which are preferably formed from hospital grade, semi-opaque or transparent, plastic tubing of approximately 22-25 mm in diameter.

The tubing section 12a is preferably about 500-750 mm long and has a mixture of fresh and carbon dioxide depleted gas flowing therethrough as indicated by arrow 14, preferably at a rate of about 80 L/min and at a pressure of 5-15 mm Hg.

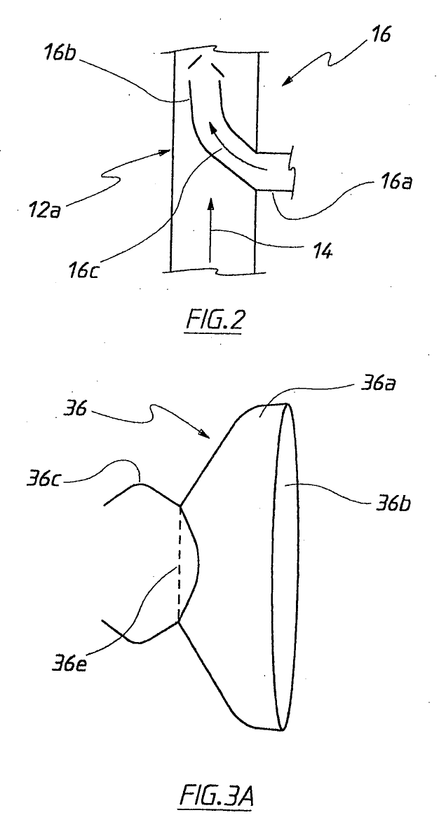

A venturi means, in the form of a jet delivery nozzle 16, receives fresh gas (eg. Oxygen) from a supply 18. The jet delivery device 16 is in gas communication with the circuit section 12a and will be described in more detail below (with reference to FIG. 2), suffice to presently say that the gas leaving the nozzle 16 causes, by virtue of entrainment due to a venturi effect, an acceleration, and thus a velocity increase, of the gas mixture 14 flowing in circuit section 12a. The gas leaves the nozzle 16 at about 4-8 L/min.

During transportation, the gas supply 18 is normally in the portable form of gas cylinders or bottles. After transportation, the gas can be supplied from cylinders or can be in the fixed form of a piped supply, as is present in most hospitals. The cylinder and piped supplies are connected to a gas blender 20 by lines 22 and 24 respectively. Suitable blenders and lines will be well known to persons skilled in the art and will not be described in further detail herein. The blender 20 has output lines 26 and 28 through which may pass only cylinder supplied gas, only pipe supplied gas or, if desired, a mixture of both. The length of the lines 22 and 24 will not influence the functioning of the device 10 and are determined by the transport environment in which the device 10 will be operated.

The line 26 connects the blender 20 to an adjustable gas flow meter 30, which provides an indication of the gas supply flowrate. Such meters are also well known to persons skilled in the art and will not be described in further detail herein. The gas leaving the meter 30 is supplied to the jet delivery nozzle 16 by an output line 32. The line 32 is one commonly used in association with such meters and is preferably not more than 50 cm in length.

The gas mixture 14 in the circuit section 12a is communicated to a uni-directional flow device in the form of a one-way valve 34. The valve 34 also has a fan therein which further assists in increasing, or at least maintaining, the velocity of the gas mixture 14 passing from the circuit section 12a to the circuit section 12b. Numerous types of suitable one way valves and fans would also be well known to a person skilled in the art.

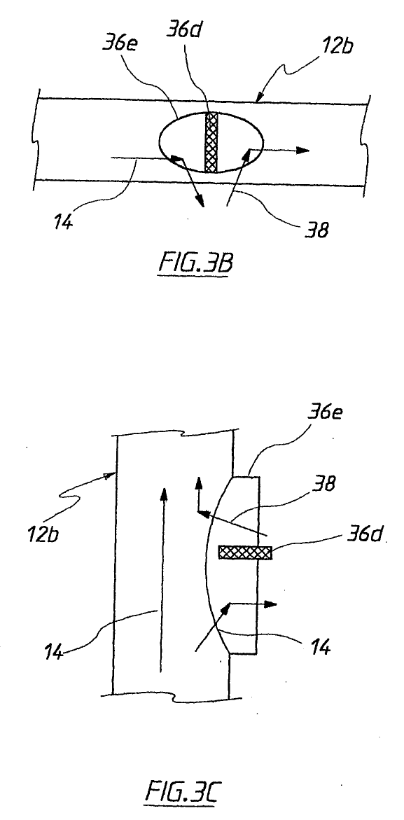

The circuit section 12b supplies the mixture 14 of fresh and carbon dioxide depleted gas to a face mask 36 placed over the patient's airways. The mask 36 shall be described in more detail below.(with reference to FIG. 3), suffice to presently say that it is able to deliver the gas mixture 14 from the circuit section 12b to the patient's airways and return only the patient's exhaled gas 38 to the circuit section 12b. Any gas flowing through circuit section 12b which is surplus to the patient's requirements flows directly through circuit section 12b also as indicated by arrow 14.

The mixture of gas streams 14 and 38 in the circuit section 12b then pass through an overpressure relief valve 41 and another one-way valve 42 into circuit section 12c. The mixture of gas streams 14 and 38 leaving the one-way valve 42 pass through circuit section 12c and through a carbon dioxide absorber and water trap 44. The gas flowing through circuit section 12d, down stream of the carbon dioxide absorber 44, is thus a mixture of fresh and carbon dioxide depleted gas, as again indicated by the arrow 14.

A gas reservoir, indicated generally by the reference numeral 45, is connected in gas communication with the circuit section 12d. The reservoir 45 is supplied with fresh gas, as indicated by arrows 43, from the output 28 of the blender 20, via a pressure reducing valve 46, a connecting line 47, an adjustable pressure regulating valve 48, a connecting line 49 and an overpressure release valve 50.

The reservoir 45 includes a compliant bag 52, a supplementary gas flow chamber 54 and chamber dividers 55. The interior of the bag 52 is in gas communication with the circuit section 12d at opening 56. The exterior of the bag 52, when sufficiently inflated, seals against the dividers 55 which blocks the gas supplied by the blender 20 to the supplementary gas flow chamber 54 from communicating with the circuit section 12d. When the patient's respiratory demands have drawn enough gas from the circuit to sufficiently deflate the bag 52 away from the dividers 55, then gas from the supplementary gas flow chamber 54 flows past the bag 52 and through one way flap valves 58, and into the circuit section 12d, as indicated by the arrows 59. This supplementary gas flow ensures that the patient's respiratory needs are always met. When the patient's respiratory needs are being satisfied by the circuit 12, then the excess gas flowing through the circuit 12 reinflates the bag 52 into sealing engagement with the dividers 55 to stop further gas communication from the supplementary gas flow chamber 54 to the circuit 12.

The bag 52 has a volume of about 2 litres and is produced from a highly compliant plastics material, for example Neoprene, so that changes in volume over the range of the patient's inhaled tidal volume (approximately 500 ml) do not substantially alter the tension in the bag wall and thus the internal pressure in the bag 52.

Referring to FIG. 2, the jet delivery nozzle 16 includes a connector 16a which is connected to the output line 32 of the meter 30. The connector 16a allows the gas from the meter 30 to be communicated to a nozzle 16b within the circuit section 12a, as indicated by arrow 16c.

Referring to FIGS. 3a to 3c, the mask 36 includes a mask shell 36a with an opening 36b for the patient's nose and mouth and a cradle 36c for connection with the circuit section 12b. The mask 36 also includes a diverter 36d within a port 36e, which connects the mask 36 to the circuit section 12b. When the patient inhales, the fresh gas flow 14 is communicated through the port 36e, aided by the diverter 36d, into the mask shell 36a and so to the patient's airways. When the patient exhales, exhaled gas 38 is drawn back through the port 36e and past the diverter 36d, into the circuit section 12b. The venturi effect of the excess fresh gas 14 flowing through the circuit section 12b assists in drawing the exhaled gas towards the ‘exhalation side’ of the diverter 36d, which minimises rebreathing.

A typical use of the device 10 will now be described. The circuit 12 is connected to the mask 36. The blender 20 is connected to the gas supply 18, which during transportation will normally be a gas cylinder filled with Oxygen. The blender 20 is also connected to the flow meter 30 and the pressure regulating valve 48. The output line 32 of the meter 30 is connected to the connector 16a of the jet delivery nozzle 16.

The required inspired oxygen concentration value is selected from the blender 20 and maximal flow rates are selected on the flow meter 30 and the mask 36 placed over the patient's face and sealingly secured in the manner well known to persons skilled in the art. At these flow rates, the circuit 12 will tend to overpressurise. Excess gas in the circuit 12 or in the bag 52 will be released through the overpressure release valve 41. With the escape of this excess gas, there will be a washout effect of the circuit 12b so that the fresh gas 14 circulating within the circuit 12 will rapidly be replaced and be predominantly that gas delivered via the flow meter 30 and containing the prescribed oxygen concentration. At that point, the flow of the fresh gas 14 can be reduced to levels that are more economical for transport and that maintain the desired level of ventilatory assistance. Excess gas in the chamber 54 will be released through overpressure release valve 50.

The settings of the overpressure release valves 41 and 50 thus determine the treatment or working pressure of the device 10.

With alterations of oxygen concentration within the circuit, a similar wash out process would be required. The more frequent such washout processes are performed, the greater the fresh gas consumption and the lesser the efficacy of the device 10 in a transport environment. However, with transport times being mostly of less than 2 hours duration (for interhospital) and less than 1 hour for primary transfers to hospitals, and with the need for frequent alterations of inspired oxygen concentration being uncommon, such wastage is minimal and made up for by the overall gas conservation properties of the device 10.

The advantages of the respiratory assist device described above are as follows. Firstly, as the device operates with a (substantially closed) gas circuit then its gas consumption is minimised thereby allowing it to operate for relatively long periods of time (eg. 1-1.5 hours when fed from a C-sized portable gas cylinder). Secondly, the device can be produced from small, light weight, generally plastic, components making it suitable for use in transport environments where weight and space must be minimised. The plastic components can also be easily sterilised between uses. Thirdly, the positive pressurisation of the gas supplied at the patient's face mask reduces patient respiratory effort, which is particularly desirable for a patient suffering respiratory trauma. Fourthly, the external face mask avoids the disadvantages associated with intubation. Fifthly, patent comfort is improved. Finally, the device allows verbal communication from the patient.

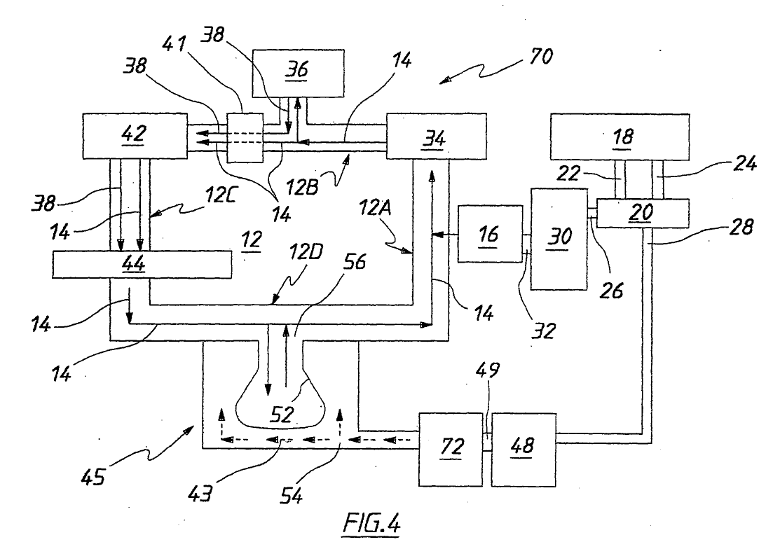

FIG. 4 shows a schematic diagram of a second embodiment of an invasive respiratory assist device 70 adapted for transport use, particularly emergency patient transportation. The device 70 is similar to the device 10 and like components to that in the first embodiment are denoted with like reference numerals in the second embodiment.

The device 70 is able to be used with intubated patients. In the device 70, the interior of the chamber 54 is sealed with respect to the exterior of the bag 52 and does not contain the dividers 55 or valves 58. Also, the chamber 54 is selectively fed pressurised gas from a ventilator 72, which is supplied with pressurised gas from the output of the blender 20 and via the valve 48. Further, the mask 36 is replaced with a tracheal tube (not shown), which are well known, and a typical Y-piece connector to connect the circuit to the tube.

The use of multi purpose connectors, and the majority of common componentry, allows removal and fitting of the components that differ between device 10 and 70 in order to easily convert between the two depending on patient need and the circumstances of use.

When the ventilator 72 is actuated, it pressurises the chamber 54. This results in the bag 52 being squeezed and thus having its contents delivered into the circuit section 12d.

The ventilator 72 can be one of several known medical transport ventilators, which are commonly dependent upon a gas supply for power. On the one hand, when such a ventilator 72 is utilised, the device 70 conserves the gas usage from the supply 18. On the other hand, the gas consumption of the ventilator 72 is wasted on squeezing the bag as that gas is not directly used to supply the patient. However, the device 70 can be used for intubated patients who only require partial assistance because of the limitation of existing transport ventilators to deliver assisted (as compared to full) ventilation. Intubated patients are otherwise currently re-sedated or given muscle paralysing drugs to be transported, and each of these actions have their own associated risks.

The devices 10, 70 also provide the choice of switching during transport from non invasive ventilation to intubation, as opposed to the present situation of intubation or no ventilatory assistance.

For intubated patients, some patients can require only continuous positive airway pressure (CPAP), only inspiratory assistance (eg. bi-level positive airway pressure), full ventilatory support (ie. mandatory) or a mixture of the latter two treatment modes.

Those that require just CPAP breathe through an intubation tube that, in adults, is usually 8 to 9 mm in diameter. The maximal achievable inspiratory gas flow rate achievable/required is much lower than that for adult patients breathing spontaneously without an endotracheal tube. Accordingly, known ventilators can match the lower flow rates required by intubated patients but have difficulty in providing the rates of flow required to provide respiratory assistance to non-intubated patients. As the device 10 is able to cope with the flow requirements of non intubated patients, the modified form of device 70 is also able to do so for intubated patients.

This is in contrast to known portable/transport ventilators which have difficulty in matching the gas flow requirements for those patients who are on bi-level or a mixture of assist and mandatory ventilation. For such patients the current safest means of ventilation is to “convert” them to mandatory only mode of ventilation. This requires sedation and/or paralysing of the patient, which each carry their own risk.

The devices 10, 70 achieve the required flow rates noted above due to the jet delivery nozzle 16, the available reservoir of gas in the bag 52 and the gas conserving benefits of the carbon dioxide absorber 44. They are thus able to improve the efficiency with which existing transport ventilators can function in an assist or assist/mandatory modes.

In another embodiment (not shown), the ventilator 72 used in the device 70 is of the type currently utilised in home bi-level ventilatory support devices. These devices are powered by mains or battery and don't deplete the fresh gas supply 18. Instead, they draw in atmospheric air, which is then used to squeeze the bag 52. The added advantage of this arrangement is the fresh gas supply 18 is conserved and the rate of gas flow it achieves within the circuit 12 is maximised.

Although the invention has been described with reference to specific embodiments, it will be appreciated by those skilled in the art that the invention may be embodied in many other forms.

For example, in another embodiment of the invention (not shown), the device is simplified by omitting the gas reservoir. However, this would result in increased fresh gas consumption and, during inspiration, the device may not be able to sustain the desired pressure level, which could lead to patient discomfort.

In other embodiments of the invention (not shown), the device also includes one or more of: a circuit pressure monitor and an associated alarm; an oxygen level analyser; a carbon dioxide analyser; a safety valve adapted to open the circuit to atmosphere upon sensing that the fresh gas supply is exhausted; and a device adapted to automatically control the fresh gas flow delivery and in response to detecting pressure changes in the circuit.

The claims defining the invention are as follows:

1. A respiratory assist device adapted for transport use, the device including the following components in a gas circuit having at least a breathable gas mixture of fresh gas and Carbon Dioxide depleted gas travelling therethrough:
a venturi means adapted to receive fresh gas from a fresh gas source and entrain the fresh gas into the gas mixture in the circuit, thereby increasing the velocity of the gas mixture in the circuit;
means to deliver the gas mixture to the patient's airways, the delivery means being downstream of the venturi means and adapted to deliver the gas mixture from the circuit to a patient's airways and return the patient's exhaled gas to the circuit; and
a Carbon Dioxide absorber downstream of the delivery means, and upstream of the venturi means, and adapted to remove Carbon Dioxide from the gas mixture in the circuit.

2. The device as claimed in claim 1, wherein the delivery means is in the form of a face mask.

3. The device as claimed in claim 1, wherein the delivery means is in the form of a tracheal tube.

4. The device as claimed in claim 1, 2 or 3, wherein the venturi means is adapted to receive the fresh breathable gas from a bottled or piped supply.

5. The device as claimed in claim 4, wherein the venturi means is adapted to receive the fresh gas via a blender and an adjustable gas flow meter.

6. The device as claimed in claim 4 or 5, wherein the fresh gas is Oxygen or Oxygen enriched.

7. The device as claimed in any one of the preceding claims, wherein the venturi means is a jet delivery nozzle.

8. The device as claimed in any one of the preceding claims, wherein the Carbon Dioxide absorber includes a water trap.

9. The device as claimed in any one of the preceding claims, further including a first unidirectional flow device in gas communication with the circuit between the venturi means and the delivery means, the first unidirectional flow device adapted to only permit gas flow from the venturi means towards the delivery means.

10. The device as claimed in claim 9, wherein the first unidirectional flow device includes a fan or other device adapted to increase the velocity of the gas mixture in the circuit.

11. The device as claimed in claim 9 or 10, wherein the device also includes a second unidirectional flow device in gas communication with the circuit between the delivery means and the Carbon Dioxide absorber, the second unidirectional flow device adapted to only permit gas flow from the delivery means towards the Carbon Dioxide absorber.

12. The device as claimed in any one of the preceding claims, further including a gas reservoir in gas communication with the circuit between the Carbon Dioxide absorber and the venturi means.

13. The device as claimed in claim 12, wherein the gas reservoir is in the form of a compliant bag.

14. The device as claimed in claim 13, wherein the interior of the bag is in gas communication with the circuit.

15. The device as claimed in claim 13 or 14, wherein the exterior of the bag is adapted to, upon sufficient inflation, block a supplemental gas flow chamber from communicating with the circuit and, in the absence of sufficient inflation, to allow the supplemental gas flow chamber to communicate with the circuit.

16. The device as claimed in claim 15, wherein the supplemental gas flow chamber is connected to the circuit via at least one third uni-directional flow device adapted to only permit gas flow from the supplemental gas flow chamber towards the circuit.

17. The device as claimed in claim 16, wherein the at least one third unidirectional flow device is a pair of flap valves.

18. The device as claimed in any one of the claims 15 to 18, wherein the supplemental gas flow chamber is supplied with gas from the blender.

19. The device as claimed in claim 18, wherein the supplemental gas flow chamber is supplied with gas via a pressure reducing valve, an adjustable pressure regulating valve and an over pressure relief valve.

20. The device as claimed in any one of the claims 13 or 14, wherein the exterior of the bag is located in a sealed chamber adapted for pressurisation by a ventilator.

21. The device as claimed in claim 20, wherein the ventilator is gas powered from the fresh gas source.

22. The device as claimed in claim 20, wherein the ventilator is electrically powered.

23. The device as claimed in claim 21 or 22, wherein the ventilator is actuated to pressurise the chamber and thereby cause the volume of the gas in the bag to be added to the circuit.

24. The device as claimed in any one of the preceding claims, further including one or more of: a circuit pressure monitor and associated alarm; an Oxygen analyser; a Carbon Dioxide analyser; a safety valve adapted to open the circuit to atmosphere upon sensing that the fresh gas supply is exhausted; and a device adapted to servo control the fresh gas flow delivery.

25. A method of providing respiratory assistance adapted for a transport environment, the method including performing the following steps in a gas circuit having at least a breathable gas mixture of fresh gas and Carbon Dioxide depleted gas travelling therethrough:
entraining fresh gas into the circuit, thereby increasing the velocity of the gas mixture in the circuit;
delivering the gas mixture, downstream of the fresh gas entrainment, from the circuit to a patient's airways and returning the patient's exhaled gas to the circuit; and
absorbing Carbon Dioxide from the circuit downstream of the patient's airways and upstream of the entrainment.

26. The method as claimed in claim 25, further including the step of receiving the fresh gas for entrainment from a bottled or piped supply.

27. The method as claimed in claim 26, further including the step of receiving the fresh gas after passing it through a blender and an adjustable gas flow meter.

28. The method as claimed in claim 25, 26 or 27, wherein the step of the entrainment is performed with a venturi means

29. The method as claimed in claim 28, wherein the step of the entrainment is performed with a jet delivery nozzle.

30. The method as claimed in any one of claims 25 to 29, further including the step of absorbing water from the circuit downstream of the patient's airways and upstream of the entrainment.

31. The method as claimed in claim 30, wherein the water is absorbed during the Carbon Dioxide absorption.

32. The method as claimed in any one of claims 28 to 31, further including the step of allowing only unidirectional gas flow in the circuit from the venturi means towards the patient's airways.

33. The method as claimed in any one of claims 25 to 32, further including the step of increasing the velocity of the breathable gas mixture in the circuit

34. The method as claimed in any one of claims 25 to 33, further including the step of allowing only unidirectional gas flow in the circuit from the patient's airways towards the Carbon Dioxide absorber.

35. The method as claimed in any one of claims 25 to 34, further including the step of providing supplemental gas flow to the circuit upon sensing that the gas flow in the circuit is not sufficient to meet the patient's respiratory needs.

36. A respiratory assist device, the device comprising the following components in a gas circuit having at least a breathable gas mixture of fresh gas and Carbon Dioxide depleted gas travelling therethrough:
a venturi for receiving fresh gas from a fresh gas source and entraining the fresh gas into the gas mixture in the circuit, thereby increasing the velocity of the gas mixture in the circuit;
an airway gas delivery assembly downstream of the venturi for delivering the gas mixture from the circuit to a patient's airways and returning the patient's exhaled gas to the circuit; and
a Carbon Dioxide absorber downstream of the mask, and upstream of the venturi, for removing Carbon Dioxide from the breathable gas mixture in the circuit.

37. The device as claimed in claim 36, wherein the delivery assembly is a face mask.

38. The device as claimed in claim 36, wherein the delivery assembly is a tracheal tube.

39. The device as claimed in claim 36, wherein the venturi is a jet delivery nozzle.

40. The device as claimed in claim 36, further including a first unidirectional flow device in gas communication with the circuit between the venturi and the airway gas delivery assembly, the first unidirectional flow device only permitting gas flow from the venturi towards the airway gas delivery assembly.

41. The device as claimed in claim 40, further including a second unidirectional flow device in gas communication with the circuit between the airway gas delivery assembly and the Carbon Dioxide absorber, the second unidirectional flow device only permitting gas flow from the airway gas delivery assembly towards the Carbon Dioxide absorber.

42. The device as claimed in claim 36, further including a gas reservoir in gas communication with the circuit between the Carbon Dioxide absorber and the venturi.

43. A method of providing respiratory assistance in a gas circuit having at least a breathable gas mixture of fresh gas and Carbon Dioxide depleted gas travelling therethrough, the method comprising the following steps:
entraining fresh gas into the circuit to increase the velocity of the gas mixture in the circuit;
delivering the gas mixture, downstream of the fresh gas entrainment, from the circuit to a patient's airways and returning the patient's exhaled gas to the circuit; and
absorbing Carbon Dioxide from the circuit downstream of the patient's airways and upstream of the entrainment.

44. The method as claimed in claim 43, wherein the step of the entrainment is performed with a venturi.

45. The method as claimed in claim 44, further including the step of allowing only unidirectional gas flow in the circuit from the venturi towards the patient's airways.

46. The method as claimed in claim 43, further including the step of increasing the velocity of the breathable gas mixture in the circuit

47. The method as claimed in claim 43, further including the step of allowing only unidirectional gas flow in the circuit from the patient's airways towards the Carbon Dioxide absorber.

48. The method as claimed in claim 43, further including the step of providing supplemental gas flow to the circuit upon sensing that the gas flow in the circuit is not sufficient to meet the patient's respiratory needs.

49. A respiratory assist device comprising:
a gas circuit having at least a breathable gas mixture of fresh gas and Carbon Dioxide depleted gas travelling therethrough;
a venturi for receiving fresh gas from a fresh gas source and entraining the fresh gas into a gas mixture in the circuit, thereby increasing the velocity of the gas mixture in the circuit;
an airway gas delivery assembly downstream of the venturi for delivering the gas mixture from the circuit to a patient's airways and returning the patient's exhaled gas to the circuit; and
a Carbon Dioxide absorber downstream of the mask, and upstream of the venturi, for removing Carbon Dioxide from the breathable gas mixture in the circuit.

50. A method of providing respiratory assistance in a gas circuit, the method comprising the following steps:
entraining fresh gas into the circuit to increase the velocity of a breathable gas mixture in the circuit, the gas mixture having the fresh gas and also Carbon Dioxide depleted gas travelling therein;
delivering the gas mixture, downstream of the fresh gas entrainment, from the circuit to a patient's airways and returning the patient's exhaled gas to the circuit; and
absorbing Carbon Dioxide from the circuit downstream of the patient's airways and upstream of the entrainment.
